(19) United States
(12) Patent Application Publication
(10) Pub. No.: US 2007/0163027 A1
(43) Pub. Date: Jul. 19, 2007

(54) Clothing with Water-Absorbent Material

(75) Inventor: Wayne Hamilton, Columbus, GA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: CHILL OUT, USA, INC.

(21) Appl. No.: 11/610,472

(22) Filed: Dec. 13, 2006

Related U.S. Application Data
(60) Provisional application No. 60/749,753, filed on Dec. 13, 2005.
(60) Provisional application No. 60/749,785, filed on Dec. 13, 2005.

(51) Int. Cl. A42B 1/04; A61F 13/15

(52) U.S. Cl. 2/171

(57) ABSTRACT

Clothing articles that contain a water-absorbent material that absorbs moisture and preferably assists in reducing body temperature and methods of using these clothing articles to reduce body temperature are described herein. The clothing articles are formed from one or more moisture wicking fabrics and include a water-absorbent material surrounded by the moisture-wicking fabric. A preferred water-absorbent material is polyvinyl alcohol material, such as a polyvinyl alcohol towel or chamois. In one embodiment, the water-absorbent material is enclosed in a pouch or pocket in the fabric, preferably the pouch or pocket is positioned to contact a surface of a user's body. In another embodiment, the water-absorbent material can be placed into a fold in the fabric or in a liner within the clothing article. A wide variety of clothing articles may be designed to contain a water-absorbent material, including, but are not limited to, hats, socks, head-bands, wrist-bands, gloves, shirts, shorts, and pants.

### CROSS-REFERENCE TO RELATED APPLICATION

This application claims benefit of and priority to U.S. Provisional Patent Application No. 60/749,753, entitled “Clothing with Built-in Cooling Properties”, filed on Dec. 13, 2005 and U.S. Provisional Patent Application No. 60/749,785, entitled “Closed Loop Clothing”, filed on Dec. 13, 2005.

### FIELD OF THE INVENTION

The present invention relates generally to clothing articles that help reduce body temperature.

### BACKGROUND OF THE INVENTION

Clothing that has been modified to reduce body temperature (“cool clothing”) has been commercially available for years. Typically, cool clothing is made of one or more fabrics that have a wicking property. The wicking property refers to the ability of the fabric to remove moisture from the skin and thereby cool the surrounding area of user's body. The fabrics typically remove moisture by allowing the moisture to evaporate. Thus, the amount of heat that can be transferred away from the skin is limited amounted of moisture that the fabric is able to remove through evaporation.

Therefore it is an object of the present invention to provide clothing articles with improved capabilities to reduce body temperature.

It is another object to provide methods for reducing body temperature, especially during exercise or in hot climates.

### SUMMARY OF THE INVENTION

Clothing articles that contain a water-absorbent material that absorbs moisture and preferably assists in reducing body temperature and methods of using these clothing articles to reduce body temperature are described herein. The clothing articles are formed from one or more moisture wicking fabrics and include a water-absorbent material surrounded by the moisture-wicking fabric. A preferred water-absorbent material is polyvinyl alcohol material, such as a polyvinyl alcohol towel or chamois. In one embodiment, the water-absorbent material is enclosed in a pouch or pocket in the fabric, preferably the pouch or pocket is positioned to contact a surface of a user's body. In another embodiment, the water-absorbent material can be placed into a fold in the fabric or in a liner within the clothing article. A wide variety of clothing articles may be designed to contain a water-absorbent material, including, but are not limited to, hats, socks, head-bands, wrist-bands, gloves, shirts, shorts, and pants.

### BRIEF DESCRIPTION OF THE DRAWINGS

FIG. 1 shows a visor containing a water-absorbent material in the sweatband.

FIG. 2A shows a side perspective view of a hat containing a water-absorbent material in the sweatband.

FIG. 2B shows a top view of the hat.

FIG. 3 shows shirt containing water-absorbent material across the shoulders and upper back portion of the shirt.

### DETAILED DESCRIPTION OF THE INVENTION

I. Definitions

The term “cool clothing” refers to clothing made from one or more fabrics that have a moisture wicking property, referred to herein as “moisture wicking fabrics”.

The term “garment” refers to an article of clothing. The term “garment” and “clothing article” are used herein interchangeably.

The term “water-absorbent material” refers to a material which absorbs at least 10 times its weight in water, or aqueous solutions.

II. Clothing Articles

The clothing articles are formed from one or more moisture wicking fabrics and include a water-absorbent material surrounded by moisture-wicking fabric(s). In some embodiments the water-absorbent material is removable from the clothing article, while in others the water-absorbent material and/or heat-transfer material is attached to or inside of the clothing article. The clothing articles are designed to increase moisture absorption and facilitate cooling of a user's body temperature.

a. Moisture-Wicking Fabrics

The clothing articles may be formed using any suitable moisture-wicking material. Moisture-wicking fabrics refer to any fabric made from a fiber that does not absorb moisture into the fiber, rather the moisture remains on the surface of the fiber and thereby promotes rapid evaporation. Suitable fabrics include polyester, polyester-based fabrics, nylon, polypropylene, polyurethane, and blends or combinations thereof. The fabrics may be formed of natural materials, such as cotton, wool, silk, and linen. Fabrics may include a mixture of natural and/or synthetic materials. Preferred fabrics include polyester and blends thereof, such as polyester-Spandex, polyester-LYCRA® (Invista), nylon and blends thereof, such as nylon-Spandex. A number of moisture-wicking fabrics have been designed and are commercially available, such as the polyester-based fabric, COOLMAX® Extreme by Invista, DRI-RELEASE® fabric by Optimer, Inc. (e.g. 88% Polyester/9% Cotton/3% Spandex; or 85% Polyester, 15% Cotton)), NANO-TEX® Coolest Comfort fabrics by Nano-Tex, Inc. (may be used with a variety of fabrics, including 100% cotton), and CLIMAGUARD®, a TEFLON®-impregnated fabric developed by ROTOFIL®.

A wide variety of different types of clothing articles may be formed. The water-absorbent material can be incorporated into work clothes, sportswear, uniforms, or ordinary street clothes. Suitable types of clothing articles include, but are not limited to shirts, shorts, pants, skirts, skorts, culottes, hats, visors, belts, gloves, headbands, and wristbands. In a preferred embodiment, the clothing articles are designed for use during exercise. However, any type of clothing article may be designed to include on or more areas for placing a water-absorbent and/or heat-transfer material.

b. Areas in the Clothing Articles for Placing a Water-Absorbent Material

The clothing articles contain one or more areas for placing a water-absorbent material, such as pockets, pouches, folds (e.g., a fold in the fabric, creating a pouch), inserts in the lining. The area for placing the water absorbent material may contain an open portion, such as on a top portion of the area. The open area may be closed using one or more fasteners. Any conventional fastener may be used, including but not limited to, snaps, clips, zippers, the strings, buttons, hooks and eyes, and hook-and-loop materials, typically available under the VELCRO® trademark. The fastener is used to close or seal the area and keep the water-absorbent material inside the area, even during vigorous exercise.

c. Labels

In one embodiment, the clothing article contains a pre-printed label, preferably on the inside of the area for placing the water-absorbent material so as not to be visible when the clothing article is worn. The label can facilitate recycling the clothing when it is no longer usable. The ink is a preferably permanent ink that will not fade over the life of the garment, enabling the wearer to send the clothing to a predetermined recycling facility. In one preferred embodiment, the clothing article includes a pre-paid postage permit from the United Postal Service or in the case of a private service, the prepaid identification from a particular carrier.

d. Representative Clothing Article

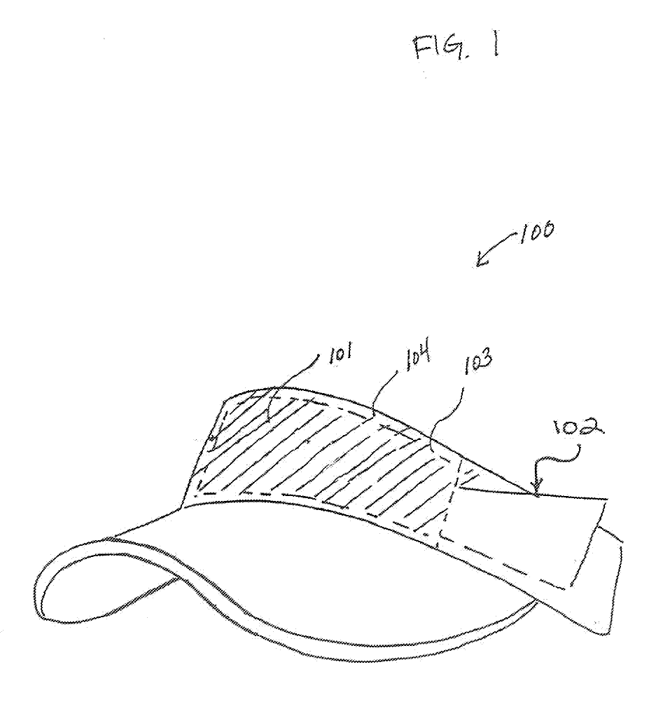

A hat or visor may be designed to include one or more areas for placing a water-absorbent material. As illustrated in FIG. 1, a water-absorbent material 102 may be inserted into a pocket or pouch 103 in a visor 100. The pocket or pouch 103 is located between the visor exterior fabric 101 and the visor headband 104. The visor exterior fabric 101 and the visor headband 104 are formed from the same or different moisture-wicking fabrics. The water-absorbent material 102 can be in the form of a towel, strip of fabric, or pad. In a preferred embodiment, in use, the water-absorbent material 102 absorbs moisture from a user's body, such as perspiration, which is removed from the exterior fabric 101 by evaporation. The removal of perspiration from the user's body surface transfers heat from the user's body to the surrounding environment.

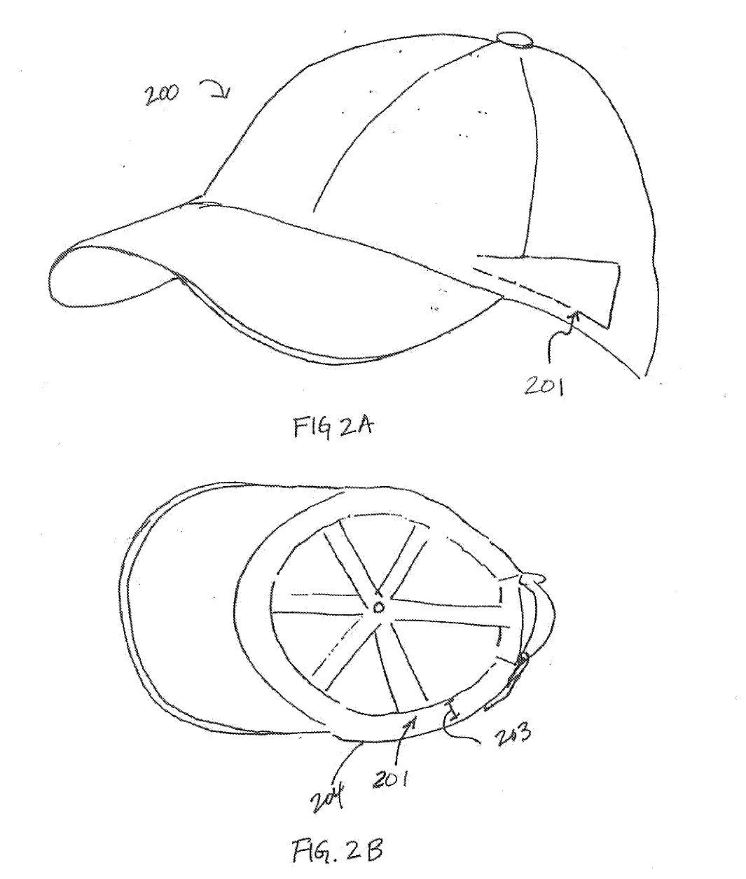

As illustrated in FIG. 2, a water-absorbent material 201 may be inserted into a gap or fold 203 in a hat 200. In this embodiment, the gap or fold 203 is located between the sweatband (not shown in figure) of the hat 200 and the exterior surface 204 of the hat 200. The sweatband of the hat 200 and the exterior surface 204 of the hat 200 are formed from the same or different moisture-wicking fabrics. The water-absorbent material 201 can be in the form of a towel, strip of fabric, or pad. In a preferred embodiment, in use, the water-absorbent material 201 absorbs moisture from a user's body, such as perspiration, which is removed from the exterior surface 204 by evaporation. The removal of perspiration from the user's body surface transfers heat from the user's body to the surrounding environment.

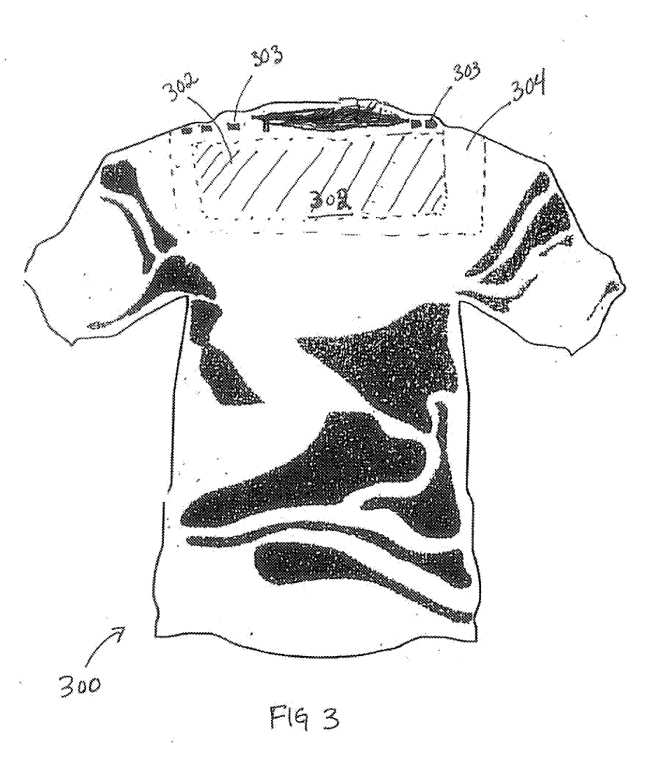

Additional articles of clothing, such as shirts, shorts, pants, skirts, skorts, culottes, etc., can be designed to include on or more areas for placing a water-absorbent material. For example, FIG. 3 depicts a shirt 300 containing a water-absorbent material 302 positioned within pouch or pocket 304 on the inside of the shirt 300. As illustrated in FIG. 3, the pouch or pocket 304 is located across the shoulders and upper back portion of the shirt. However, the pouch or pocket may be located in an alternative or additional location(s). The pouch or pocket 304 optionally includes a fastener 303 for closing or sealing the pocket 304 to keep the water-absorbent material 302 inside the pouch or pocket 304. Fastener 303 can be any conventional fastener.

In another embodiment, a headband may be designed to contain a water-absorbent material inside the headband material. A fold or pocket can be formed between the exterior and interior surfaces of the headband. The headband is formed of a moisture-wicking material, which allows for the transfer of moisture through a first, inner layer of the headband material, to the water-absorbent material, and then through the water absorbent material to a second, outer later of the headband material. Typically, the water-absorbent material can be removed from the headband or other clothing article, stored separately from the clothing article and/or replaced as needed. In one embodiment, the water-absorbent material is sewn inside the headband material and is not easily removable.

III. Water-Absorbent Materials

The water-absorbent material absorbs at least approximately 10 times in weight in water, or aqueous solutions. In a preferred embodiment, the water-absorbent material absorbs at least approximately 12 times in weight (dry) in water or aqueous solutions. Preferably the material absorbs up to 12 times its weight in water or aqueous solutions without feeling wet to a user. The water-absorbent materials do not dissolve in water at standard temperatures and pressures. The water-absorbent material is typically in the form of a towel, pad or strip of material. Any suitable thickness may be used, preferably the water-absorbent material does not significantly increase the thickness of the overall clothing articles when placed in the pouch, fold or pocket. Thinner water-absorbent materials are generally preferred. Typical thicknesses range from up to 1 cm to up to 4 cm and between about 0.5 cm and about 4 cm. In one preferred embodiment, the water-absorbent material typically has a thickness ranging from 2 cm to 4 cm.

The water-absorbent material can be formed of a polymer or non-polymeric substance, such as silica. The polymer can be a homopolymer, a copolymer, or a polymer blend or polymer composite. Suitable polymers which form can form water-absorbent materials, but are not limited to, synthetic or natural polymers. Examples of synthetic polymers include polyvinyl alcohol, polyacrylic and polymethacrylic acid polymers, cellulose derivatives such as hydroxypropyl cellulose, polyethyleneglycol polymers, copolymers and block copolymers, and other water swellable, biocompatible polymers. Examples of natural polymers include collagen, hyaluronic acid, gelatin, albumin, polysaccharide, and derivatives thereof. Natural polymers can also be of the type isolated from various plant materials such as psyllium.

Structurally, the water-absorbent polymeric materials are three-dimensional macromolecular configurations. they are produced through several methods: a) synthesis from monomers (cross-linking polymerization); b) synthesis from polymers and polymerization auxiliary (grafting and cross-linking polymerization); c) synthesis from polymers and non-polymerization auxiliary (cross-linking polymers); d) synthesis from polymers with energy sources (cross-linking polymers without auxiliaries) and e) synthesis from polymers (cross-linking by reactive polymer-polymer intercoupling). The raw materials and technology used in synthesis are main factors in the creation of water-absorbent materials' key properties and their range of applications.

There are a known number of methods for obtaining high purity water-absorbent materials with three-dimensional polymeric configurations and with potential applications in pharmaceutical and/or medical field: a) chemical methods: ionic and/or coordinative intercomplexing (i.e. U.S. Pat. No. 4,570,629 to Widra and U.S. Pat. No. 5,153,174 to Brand et al.); cross-lining with oligomers or reactive polymers that have reactive groups with double bonds or rings (i.e. U.S. Pat. No. 5,489,261 Franzblau et al. and U.S. Pat. No. 5,863,984 to Doillon et al.); cross-linking with radiation (i.e. U.S. Pat. No. RE33,997 to Kuamz et al.; U.S. Pat. No. 4,264,155 to Miyata; and U.S. Pat. No. 5,948,429 to Bell et al.); and b) physical methods: cross-linking with microwaves (i.e. U.S. Pat. Nos. 5,859,077 and 6,168,762 to Reichman et al.); freeze-drying (i.e. U.S. Pat. No. 5,676,967 to Williams et al. and U.S. Pat. No. 5,869,080 to McGregor et al.); and dehydrothermo-crosslinking (i.e. U.S. Pat. No. 4,837,285 to Berg et al.; U.S. Pat. No. 4,950,485 to Akhtar et al.; and U.S. Pat. No. 4,971,954 to Brodsky et al.). U.S. Pat. No. 6,013,587 to Troung et al. discloses absorbent nonwoven articles formed of a polyvinyl alcohol fibers and a binder.

Dehydrothermal-crosslinking, as with the other physical methods for obtaining three-dimensional structures, eliminates the risk of toxic effects that can be produced by secondary products of the reaction or energy state modification of the reaction product (in which appear new types of covalent, ionic or coordinative bonds), which can occur in the activation of some chemical process. Moreover, compared with freeze-drying or cross-linking via microwaves, dehydrothermo-crosslinking offers many more possibilities to regulate the structural characteristics of the resulting three-dimensional networks (i.e. Scotchford C. A., Casone G. D., Owens S., Gusti P., “Osteoblast responses of collagen-PVA bioartificial polymers in vitro: the effects of cross-linking method and collagen content”, Biomaterials 19,1-11, 1998; Guinchedi P., Genta I., Conti B., Muzzarelli R. A. A., Conti B., Biomaterials 19,157-161, 1998). The hydrogels based on collagenic biopolymers obtained by dehydrothermo-crosslinking, however, do not have high absorption capacities.

In the preferred embodiment, the water-absorbent material is a polyvinyl alcohol (PVA) material, such as a PVA towel, commonly referred to by swimmers as a “Sammy towel”. The PVA towel is typically about 2 to 4 cm thick and may be cut to the desired shape and size. The material behaves in a sponge-like manner. it can hold a minimum of 10 times its weight in aqueous liquids without feeling wet to a user. When wet, the temperature of the towel is typically lower than the outside air. Thus, the PVA towel may also be used to cool the user. PVA towels are typically machine washable. PVA towels are commercially available from a variety of suppliers. PVA towels are also sold under a variety of trademarks, including the CHILLY PAD® sports towel (Guntersville Breathables, Inc.) and the “Sammy towel” or “PVA Shammy”.

IV. Methods of Use

The clothing articles described herein can be used to absorb moisture and reduce body temperature in a specific region of the body or systemically, for example during exercise, strenuous work, or in climates having a temperature above about 75° F. The garments can be worn as typical clothing, as sporting attire, or as an accessory such as a hat, headband, belt or wristband. It will be appreciated that one or more water-absorbent materials can be used in a single clothing article.

The clothing articles are designed to increase moisture absorption and facilitate cooling of a user'body temperature. When worn by an individual, the clothing articles remove perspiration from the surface of the user's body that is in contact with a first, inner layer of moisture-wicking fabric. The moisture is then absorbed by the water-absorbent material. In a preferred embodiment, at least a portion of the moisture is transferred from the water-absorbent material to a second, outer layer of the moisture-wicking fabric. the outer layer may contain the same fabric or a different fabric than the inner layer. The moisture is removed from the outer layer of the moisture-wicking fabric by evaporation. In a preferred embodiment, when the water-absorbent material absorbs the moisture, the temperature of the pad is decreased relative to the ambient environment. This increases the cooling effect of the clothing article.

When worn by an individual, the clothing articles are particularly useful at preventing perspiration from running down a user's face, hands, back, or body. Thus, when a headband, hat or visor as described herein is worn, the clothing article helps prevent a user's glasses from sliding off of his or her face. And, when a wristband as described herein is worn, the clothing article allows a user to maintain his grip on a racket or ball, or other device or instrument.

When the clothing article is not being used, the water-absorbent material may be removed from the clothing article and stored separately. For PVA materials, the material is typically moistened, such as by washing or rinsing it, and stored in a sealed bag to retain its moisture. Prior to use, the PVA material is moistened and placed in the clothing article.

For embodiments in which the water-absorbing material is located within the clothing article and is not easily removable therefrom, the clothing article is typically moistened prior to use. This will assist in the cooling properties for the water-absorbent material, such as a PVA material, and reduce the stiffness for the water-absorbent material prior to use.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A clothing article comprising
a first moisture-wicking fabric, wherein one surface of the fabric is designed to be in contact with a body portion in use;
a water-absorbent material,
a second moisture-wicking fabric, wherein the first and the second moisture wicking fabrics are the same or different materials, and
at least one area connected to the first moisture-wicking fabric, wherein the area is designed to contain the water-absorbent material.

2. The clothing article of claim 1, wherein the water-absorbent material comprises polyvinyl alcohol.

3. The clothing article of claim 1, wherein the water-absorbent material is in a form selected from the group consisting of a towel, a strip of material, and a pad.

4. The clothing article of claim 1, wherein the first and the second moisture wicking fabrics are the same material.

5. The clothing article of claim 1, wherein the first and the second moisture wicking fabrics are the different materials.

6. The clothing article of claim 1, wherein the first and second materials are selected from the group consisting of polyester, polyester-based fabrics, nylon, polyurethane, and blends or combinations thereof.

7. The clothing article of claim 1, in a form selected from the group consisting of headbands, visors, hats, shirts, shorts, pants, wristbands, gloves.

8. The clothing article of claim 1, wherein the area designed to contain the water-absorbent material is in a form selected from group consisting of pouches, pockets, and folds.

9. The clothing article of claim 1, wherein area designed to contain the water-absorbent material comprises at least one fastener.

10. The clothing article of claim 7, in the form of a headband, wherein the area designed to contained the water-absorbent material is in the form of a fold in the first or second fabric.

11. The clothing article of claim 10, wherein the first and second fabrics are the same continuous material.

12. The clothing article of claim 7, in the form of a visor or hat, wherein the area designed to contain the water-absorbent material is in the form of a pocket or pouch between the first fabric and the second fabric.

13. The clothing article of claim 7, in the form of a shirt, shorts or pants, wherein the area designed to contain the water-absorbent material is in the form of a pocket or pouch between the first fabric and the second fabric.

14. The clothing article of claim 7, further comprising a label in permanent ink, wherein the label identifies the type or location of the recycling facility that can be used to recycle the clothing article.

15. A method for reducing a user's body temperature comprising applying a clothing article to the user's body, wherein the clothing article comprises
a first moisture-wicking fabric, wherein one surface of the fabric is designed to be in contact with a body portion in use;
a water-absorbent material,
a second moisture-wicking fabric, wherein the first and the second moisture wicking fabrics are the same or different materials, and
at least one area connected to the first moisture-wicking fabric, wherein the area comprises the water-absorbent material.

16. The method of claim 15, wherein the user's body temperature is reduced by wicking moisture away from the surface of the user's body through the first fabric, and absorbing the moisture in the water-absorbent material.

17. The method of claim 16, further comprising transferring at least a portion of the water from the water-absorbent material through the second fabric, and evaporating the water from the surface of the second fabric.
